(19) United States
(12) Patent Application Publication
(10) Pub. No.: US 2006/0116519 A1
(43) Pub. Date: Jun. 1, 2006

(54) Synthesis of 5-bromo-4-methyl-pyridin-3-ylmethyl)-ethyl-carbamic acid tert-butyl ester

(75) Inventors: Chunrong Ma, San Diego, CA (US); Qingping Tian, San Diego, CA (US)

Correspondence Address: AGOURON PHARMACEUTICALS, INC., 10777 SCIENCE CENTER DRIVE, SAN DIEGO, CA, 92121, US

(73) Assignee: AGOURON PHARMACEUTICALS, INC.

(21) Appl. No.: 11/238,922

(22) Filed: Sep. 28, 2005

Related U.S. Application Data
(60) Provisional application No. 60/629,144, filed on Nov. 17, 2004.

(51) Int. Cl. C07D 213/26

(52) U.S. Cl. 546/329

(57) ABSTRACT

The present invention relates to novel synthetic methods for the preparation of intermediates of 3{5-[3-(4,6-Difluoro-1H-benzoimidazol-2-yl)-1H-indazol-5-yl]4-methyl-pyridin-3-yl methyl}-ethyl-amine.

This application claims the benefit of U.S. Provisional Application No. 60/629,144, filed Nov. 17, 2004, the contents of which are herein incorporated in its entirety.

### FIELD OF THE INVENTION

This invention relates to novel synthetic routes for the preparation of intermediates useful in the preparation of {5-[3-(4,6-Difluoro-1H-benzoimidazol-2-yl)-1H-indazol-5-yl]-4-methyl-pyridin-3-ylmethyl}-ethyl-amine.

### BACKGROUND OF THE INVENTION

The present invention relates to a new stereoselective method for the preparation of key intermediates towards the synthesis of a protein kinase inhibitor.

The compound {5-[3-(4,6-Difluoro-1H-benzoimidazol-2-yl)-1H-indazol-5-yl]-4-methyl-pyridin-3-ylmethyl}-ethyl-amine (also referred to as “Compound 1”), 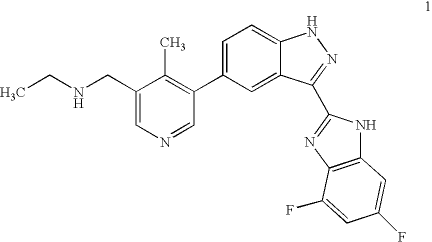
as well as pharmaceutically acceptable salts and solvates thereof, is described in U.S. patent application Ser. No. 10/866,059, filed 10 Jun. 2004, the disclosure of which is hereby incorporated in its entirety. This compound is a protein kinase inhibitor and represents a synthetic, small molecule inhibitor capable of modulating cell cycle control.

A method of preparing Compound 1 is disclosed as Example 1 of U.S. patent application Ser. No. 10/866,059. This method is inefficient however, in terms of producing large quantities of Compound 1 for clinical and commercial scale-up. In particular, the preparation of a key intermediate in the method involves two cryogenic reactions and generates unwanted side-products, all of which are undesirable for commercial production. In addition, the current synthetic route for preparing Compound 1 involves several inefficient and wasteful recrystallization steps. Therefore, a preparation of the CDK inhibitor {5-[3-(4,6-Difluoro-1H-benzoimidazol-2-yl)-1H-indazol-5-yl]-4-methyl-pyridin-3-ylmethyl}-ethyl-amine which is cost-efficient, scaleable and productive is highly desirable.

### SUMMARY OF THE INVENTION

The present invention is directed to method of making a key intermediate 1b useful in the synthesis of the CDK inhibitor {5-[3-(4,6-Difluoro-1H-benzoimidazol-2-yl)-1H-indazol-5-yl]-4-methyl-pyridin-3-ylmethyl}-ethyl-amine (“Compound 1”). In particular, the present invention is directed to a method of preparing a compound having the formula 1b 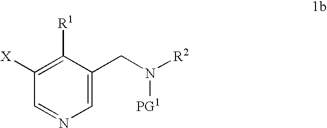
wherein X is a halo;
PG¹ is a protecting group selected from tert-butoxycarbonyl (“BOC”), benzyloxycarbonyl (“CBZ”), CH₂C₆H₅ (“Bn”), 2-(trimethylsilyl)ethoxymethyl (“SEM”), tetrahydropyran (“THP”), trimethylsilyl (“TMS”);
R¹ and R² are, independently, alkyl, alkenyl, alkynyl, cycloalkyl or aryl;
comprising the step of reacting a compound of formula 6 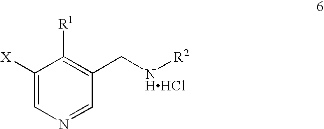
   with PG¹ and a base in a suitable solvent. In one aspect of this step, the base selected from aqueous NaOH, 4-dimethylaminopyridine (“DMAP”), N,N-diisopropylethylamine (“EtN(i-Pr)₂”), or triethylamine (“NEt₃” or “TEA”).

In another aspect of this step, the suitable solvent is selected from tetrahydrofuran (“THF”), methylene chloride (“CH₂Cl₂”), methyl tert-butyl ether (“MTBE”), toluene or a combination thereof. In yet another aspect of this step, the reaction is performed at a temperature of about 0° to 15° C. In still another aspect of this step, the reaction is performed for a minimum of at least 1 h. In a specific embodiment of this step, X is Br, R¹ is methyl, R² is ethyl, PG¹ is BOC, the suitable solvent is THF, and the reaction is performed for at least 2 hours.

The present invention is further directed to a step of preparing a compound of formula 6, by reacting a compound of formula 5 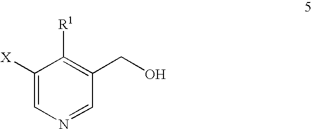
with R²NH₂, an acid chloride and a base in a suitable solvent. In one aspect of this step, the reaction is performed in the presence of a suitable solvent selected from THF, CH₂Cl₂, MTBE, or toluene or a combination thereof. In another aspect of this step, the acid chloride is methanesulfonyl chloride (“MsCl”) or p-toluenesulfonyl chloride (“TosCl”). In yet another aspect of this step, the base is selected from aqueous NaOH, DMAP, NEt₃, or EtN(i-Pr)₂. In still another aspect of this step, the reaction step is performed at a temperature from about 0° to −20° C. In another aspect of the invention, the reaction is performed for at least 0.5 hr. In one specific embodiment, the suitable solvent is THF, the acid chloride is MsCl and the base is NEt₃, and the reaction is performed for at least one hour.

The present invention is further directed to a step of preparing a compound of formula 5 by reacting a compound of formula 4 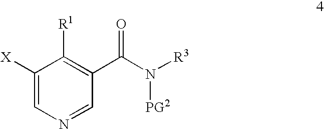
wherein PG² is a protecting group selected from BOC, CBZ, Bn, SEM, THP or TMS;
R³ is selected from alkyl, alkenyl, alkynyl, cycloalkyl or aryl;
with a reducing agent and an alcohol. In one aspect of this invention, the step requires a reducing agent selected from NaBH₄, LiBH₄ or LiAlH₄. In another aspect of the invention, the reaction is performed in the presence of an alcohol selected from methanol, ethanol, or a sequential combination thereof. In one aspect of the invention the reaction was performed at a temperature of 0°-20° C. In another embodiment, the reaction was performed for at least 10 hrs. In a specific embodiment of this step, R³ is ethyl, PG² is BOC, the reducing agent is NaBH₄ and the alcohol is a sequential succession of ethanol followed by methanol, and the reaction is performed overnight.

The present invention is further directed to a step of preparing a compound of formula 4 by reacting a compound of formula 3 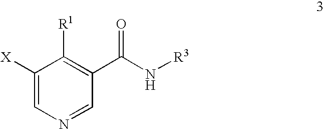
with PG² and a base in a suitable solvent. In one aspect of this step, the suitable solvent in this step is selected from THF, CH₂Cl₂, MTBE, toluene or a combination of solvents. In another aspect of this step, the base is selected from aqueous NaOH, DMAP, NEt₃ or EtN(i-Pr)₂. In yet another aspect of this step, the reaction is performed at a temperature of about 10°-30° C. In still another aspect of this step, the reaction is performed for at least 10 hours. In one specific embodiment of this step, the suitable solvent is THF, the base is DMAP, and the reaction is performed overnight.

The present invention is further directed to a step of preparing a compound of formula 3 by reacting a compound of formula 2 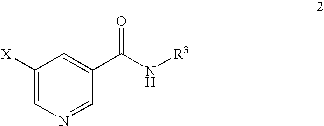
with an alkylating agent and an oxidizing reagent in a suitable solvent. In one aspect of this step, the alkylating agent is R¹MgCl, R¹Li, (R¹)₂CuLi or R¹ZnCl. In a further aspect of this step, the alkylating agent is a methylating agent selected from CH₃MgCl, CH₃Li, (CH₃)₂CuLi or CH₃ZnCl. In another aspect of this step, the suitable solvent is selected from THF, CH₂Cl₂, MTBE, toluene or a combination thereof. This invention may further comprise a step of adding a quenching solvent to quench the reaction of compound 2 with the alkylating agent. The quenching solvent is preferably aqueous ammonium chloride or an alcohol selected from methanol, ethanol, or a sequential combination thereof. In yet another aspect of this step, the oxidizing reagent is selected from N-bromosuccinimide (“NBS”) or N-chlorosuccinimide (“NCS”). In still another aspect of this step, the reaction occurs at a temperature of about 0° to 20° C. In still another aspect of this step, the reaction occurs for at least 0.5 hours. In one specific embodiment of this step, the methylating agent is CH₃MgCl, the oxidizing reagent is NBS, the suitable solvent is THF, the quenching solvent is aqueous ammonium chloride and the reaction is performed for at least 3 hours.

The present invention is further directed to a step of preparing a compound of formula 2 by reacting a compound of formula Q 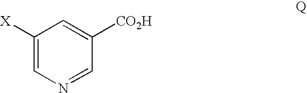
with R³NH₂ and 1,1′-carbonyldiimidazole in a suitable solvent. In one aspect of this invention the suitable solvent may be selected from THF, CH₂Cl₂, MTBE, toluene or a combination thereof. In one aspect of this step, the reaction is performed at a temperature of about 0° to 20° C. In a further aspect of this step, the reaction is performed for at least 20 minutes. In one specific embodiment, the suitable solvent is THF and the reaction is performed for at least one hour.

The term “halo”, as used herein, unless otherwise indicated, means fluoro, chloro, bromo or iodo. Preferred halo groups are fluoro, chloro and bromo.

The term “alkyl” refers to a saturated monovalent aliphatic radicals having straight, cyclic or branched moieties. Examples of alkyl radicals useful in the invention include C₁-C₆ alkyl such as methyl, ethyl, n-propyl, isopropyl, n-butyl, isobutyl, sec-butyl, tert-butyl, tert-amyl, pentyl, hexyl, as well as heptyl, octyl and the like.

The term “alkenyl” refers to unsaturated aliphatic moieties having at least one carbon-carbon double bond and including E and Z isomers of said alkenyl moiety. The term also includes cycloalkyl moieties having at least one carbon-carbon double bond wherein cycloalkyl is as defined above. Examples of alkenyl radicals include ethenyl, propenyl, butenyl, 1,4-butadienyl, cyclopentenyl, cyclohexenyl and the like.

The term “alkynyl” refers to an unsaturated aliphatic moieties having at least one carbon-carbon triple bond and includes straight and branched chain alkynyl groups. Examples of alkynyl radicals include ethynyl, propynyl, butynyl and the like.

The term “alcohol”, as used herein, unless otherwise indicated, refers to R—OH groups. Examples of alcohol groups used in the invention include lower alcohols, such as C₁-C₆ alkyl-OH groups such as methanol, ethanol, propanol and the like.

The term “aryl” refers to an aromatic group which has at least one ring having a conjugated pi electron system and includes a carbocyclic aryl, heterocyclic aryl and biaryl groups. The term “aromatic” refers to compounds or moieties comprising multiple conjugated double bonds.

The term “cycloalkyl” refers to saturated monovalent aliphatic radicals having cyclic configurations, including monocyclic, bicyclic, tricyclic, and higher multicyclic alkyl radicals (and, when multicyclic, including fused and bridged bicyclic and spirocyclic moieties) wherein each cyclic moiety has from 3 to about 8 carbon atoms. Examples of cycloalkyl radicals include cyclopropyl, cyclobutyl, cyclopentyl, cyclohexyl and the like.

The term “suitable solvent” includes solvents from hydrocarbons and ethers, preferably tetrahydrofuran, CH₂Cl₂, methyl tert-butyl ether, and toluene.

The term “oxidizing reagent” include any oxidizing agents including methylsulfoxide-based Swern-type oxidizing agents, chromium-based oxidizing agents, and iodine-based oxidizing agents such as Dess-Martin periodinane or a Swern-type oxidizing agent such as pyridine-SO₃ complex in the presence of Hunig's base. Preferred oxidizing reagents of the invention include the N-bromosuccinimide, and N-chlorosuccinimide.

The term “reducing agent” include any known reducing agents, including silanes, such as polymethylhydrosiloxane (PMHS), trichlorosilane, hexachlorodisilazane, or phenyltrisilane, optionally in the presence of catalysts which comprise monomeric zinc compounds, complexed by basic ligands such as amines, polyamines, aminoalcohols, amine oxides, amides, phosphoramides, etc. The reducing agents can also be a hydride such as LiAlH₄, NaBH₄, or LiBH₄. The reducing agents can also be hydrogen gas in the present of a metal catalyst. Preferably the reducing agent is NaBH₄, LiBH₄ or LiAlH₄.

The term “alkylating agent” refer to agents capable of deprotonation. Alkylating agents include alkylmagnesium halides such as R—MgCl, as well as RLi, R₂CuLi or RZnCl wherein R is selected from any alkyl, alkenyl, alkynyl, cycloalkyl or aryl. In a particular embodiment, the alkylating agent is a methylating agent selected from CH₃MgCl, CH₃Li, (CH₃)₂CuLi or CH₃ZnCl.

The term “base” in the present invention refers to both inorganic and organic bases such as NaOH, KOH or R—NH₂, including but not limited to DMAP, 4-dimethylaminopyridine, N,N-diisopropylethylamine and triethylamine.

The term “acid chloride” in the present invention refers to a suitable agent, such as methanesulfonyl chloride (“MsCl”), p-toluenesulfonyl chloride (“TosCl”), trifluoromethanesulphonate chloride (“TfCl”), which can convert a hydroxyl group (—OH) to a good leaving group. Preferred acid chlorides useful in the invention include MsCl and TosCl.

In the following examples and claims, “Me” means methyl, “Et” means ethyl, “CDI” means to 1,1′-Carbonyidiimidazole, “BOC” means tert-butoxycarbonyl, “CBZ” means benzyloxycarbonyl, “SEM” means 2-(trimethylsilyl)ethoxymethyl, “THP” means tetrahydropyran, “TMS” means trimethylsilyl, “THF” means tetrahydrofuran, “CH₂Cl₂” refers to methylene chloride, “MTBE” means methyl tert-butyl ether, “DMAP” means 4-dimethylaminopyridine, “EtN(i-Pr)₂” means N,N-diisopropylethylamine, “NEt₃” or “TEA” means triethylamine, “NBS” means N-bromosuccinimide, “NCS” means N-chlorosuccinimide, and “EtOAc” means ethyl acetate, “DHP” means dyhydropyran, “DMA” means N,N-dimethylacetamide, “h” means hours, “m” or “min” means minutes, “TsOH” means p-toluenesulfonic acid, “TFA” means trifluoroacetic acid, “LDA” means lithium diisopropylamide, “n-BuLi” means n-butyllithium.

### EXAMPLES

The following examples are given to illustrate the invention, but should not be considered as limitations of the invention. Unless otherwise indicated, all temperatures are set forth in degrees Celsius and all parts and percentages are by weight.

### Comparative Example 1

### Preparation of Compound 1

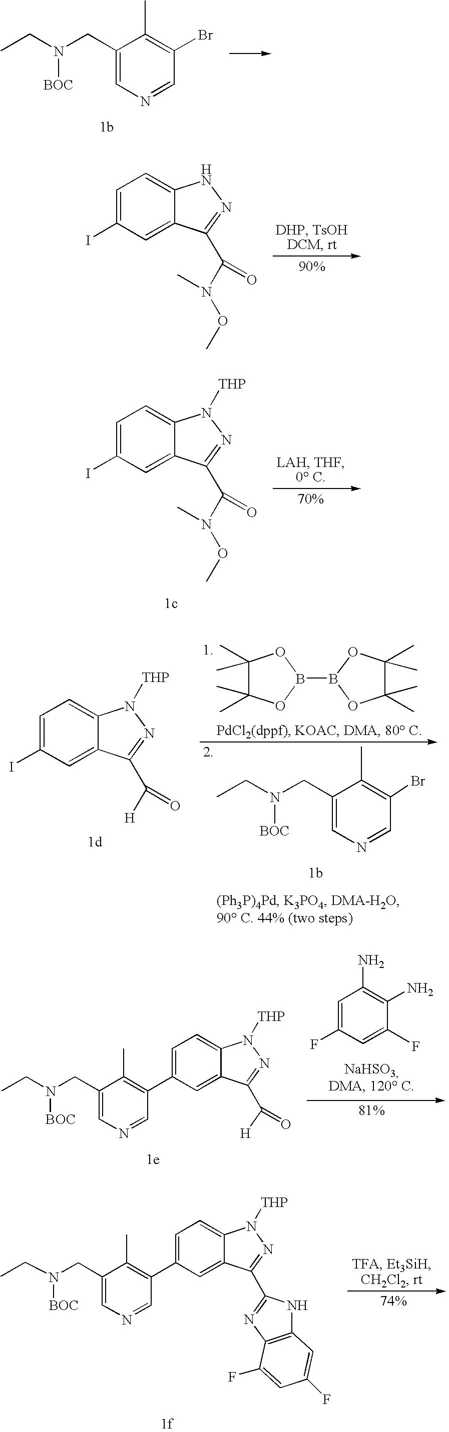

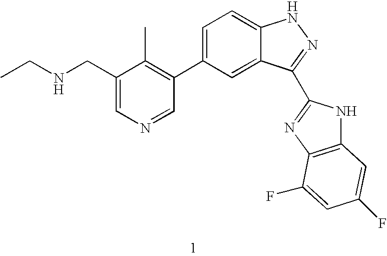

(a) Intermediate 1c—5-lodo-1-(tetrahydro-pyran-2-yl)-1H-indazole-3-carboxylic acid methoxy-methyl-amide

5-Iodo-1H-indazole-3-carboxylic acid methoxy-methyl-amide [for the preparation of this compound see: Reich, S. R.; Bleckman, T. M.; Kephart, S. E.; Romines, W. H.; Wallace, M. B., U.S. Pat. No. 6,555,539 B2, Apr. 29, 2003.] was alkylated with dihydropyran according to the method of Sun, et. al. [Sun, J.-H.; Teleha, C. A.; Yan, J.-S.; Rogers, J. D.; and Nugiel, D. A., J. Org. Chem. 1997, 62, 5627], affording amide 1c (typically >90%) as an off-white powder: ¹H NMR (DMSO-d₆) δ 8.37 (s, 1H), 7.74 (dd, 8.8 Hz, 1H), 7.68 (d, J=8.8 Hz, 1H), 5.97 (dd, J=2.3, 9.0 Hz, 1H), 3.88 (m, 2H), 3.79 (s, 3H), 3.42 (s, 3H), 2.35 (m, 1H), 2.03 (m, 2H), 1.75 (m, 1H), 1.58 (m, 2H).

(b) Intermediate 1d—5-lodo-1-(tetrahydro-pyran-2-yl)-1H-indazole-3-carbaldehyde

Lithium aluminum hydride (1.2 equiv.) is added portionwise to a cooled (<5° C.) solution of amide 1c (1.0 equiv.) in THF. Stirring is continued at <5° C. until the reaction is complete, typically 30 minutes. The reaction was quenched by the slow addition of ethyl acetate at <5° C., and the whole mixture poured into 0.4 N NaHSO₄. The organic layer was washed with brine, dried over magnesium sulfate, concentrated, and purified by silica gel chromatography to give aldehyde 1d (typically ˜70%) as an off-white powder: ¹H NMR (CDCl₃) δ 10.15 (s, 1H), 8.47 (s, 1H), 7.82 (dd, J=1.5, 8.7 Hz, 1H), 7.78 (d, J=8.5 Hz, 1H), 6.04 (dd, J=2.3, 9.28 Hz, 1H), 3.85 (m, 2H), 2.35 (m, 1H), 2.05 (m, 2H), 1.76 (m, 1H), 1.60 (m, 2H).

(c) Intermediate 1e—Ethyl-{5-[3-formyl-1-(tetrahydro-pyran-2-yl)-1H-indazol-5-yl]-4-methyl-pyridin-3-ylmethyl}-carbamic acid tert-butyl ester

Iodoindazole Id (3.56 g, 10.0 mmol), bis(pinacolato)diboron (2.79 g, 11 mmol), potassium acetate (2.74 g, 30 mmol) and [1,1′-bis(diphenylphosphino)-ferrocene]

dichloropalladium(II)complex with dichloromethane (245 mg, 0.3 mmol) were dissolved in N,N-dimethylacetamide (60 mL). The solution was degassed by evacuating (until the solvent begins to bubble) and purging with Argon (3 cycles), then heated in an 80° C. oilbath for 2 hours. After cooling slightly (to ˜50° C.), a solution of bromopyridine 1b (3.62 g, 11 mmol) in N,N-dimethylacetamide (40 mL) was added, followed by deionized water (10 mL) and potassium phosphate (3.18 g, 15 mmol). The solution was degassed, tetrakis(triphenylphosphine) palladium (0) (347 mg, 0.3 mmol) added, and degassed again. The mixture was stirred in a 90° C. oilbath for 4.5 hours. After cooling to room temperature, the mixture was diluted with ethyl acetate (300 mL), washed with deionized water (150 mL), and saturated sodium chloride (100 mL). The organic layer was dried over magnesium sulfate, filtered, and concentrated to a crude red-black oil (9.43 g). Purification by silica gel chromatography (eluting with 50-100% ethyl acetate in hexanes) afforded coupled product 1e (2.9462 g) as an orange oil. ¹H NMR of this product showed it was contaminated with ˜1 equivalent of pinacol. Trituration from hexanes afforded pure 1e (2.0853 g, 44%) as a fine yellow powder: ¹H NMR (CDCl₃) δ 10.25 (s, 1H), 8.39 (s, 1H), 8.34 (s, 1H), 8.22 (s, 1H), 7.74 (d, J=8.7 Hz, 1H), 7.38 (dd, J=1.5, 8.5 Hz, 1H), 5.88 (dd, J=2.8, 9.2 Hz, 1H), 4.53 (s, 2H), 4.03 (m, 1H), 3.81 (m, 1H), 3.24 (brs, 2H), 2.60 (m, 1H), 2.18 (s, 3H), 2.15 (m, 2H), 1.77 (m, 1H), 1.65 (m, 2H), 1.47 (s, 9H), 1.09 (t, J=7.0 Hz, 1H).

(d) Intermediate 1f—{5-[3-(4,6-Difluoro-1H-benzoimidazol-2-yl)-1-(tetrahydro-pyran-2-yl)-1H-indazol-5-yl]-4-methyl-pyridin-3-ylmethyl}-ethyl-carbamic acid tert-butyl ester

Aldehyde 1e (2.05 g, 4.28 mmol), 1,2-diamino-3,5-difluorobenzene (617 mg, 4.28 mmol) and sodium bisulfite (891 mg, 8.57 mmol) were dissolved in N,N-dimethylacetamide (43 mL) and heated in a 120° C. oilbath for 21 hours. After cooling to room temperature, the mixture was diluted with ethyl acetate (100 mL) and washed with half-saturated aqueous sodium chloride solution (75 mL, a 1:1 mixture of deionized water and saturated aqueous sodium chloride solution). The aqueous layer was back-extracted with ethyl acetate (2×100 mL). All the organic extracts were combined, dried over magnesium sulfate, and concentrated to a brown tar (3.39 g). This crude material was purified by silica gel chromatography (eluting with a gradient of 70% to 100% ethyl acetate in hexanes), to give benzoimidazole product If (2.11 g, 81%) as a tan foam: ¹H NMR (CD₃OD) δ 8.46 (s, 1H), 8.41 (s, 1H), 8.29 (s, 1H), 7.87 (d, J=8.6 Hz, 1H), 7.46 (dd, J=1.3, 8.6 Hz, 1H), 7.13 (m, 1H), 6.84 (m, 1H), 5.99 (dd, J=2.3, 9.9 Hz, 1H), 4.60 (s, 2H), 4.01 (m, 1H), 3.86 (m, 1H), 3.32 (m, 2H, obscured by solvent peak) 2.67 (m, 1H), 2.28 (s, 3H), 2.18 (m, 2H), 1.89 (m, 1H), 1.73 (m, 2H), 1.47 (s, 9H), 1.13 (t, J=7.1 Hz, 1H). Anal. (C₃₃H₃₆F₂N₆O₃·0.4 H₂O) C, H, N, F.

(e) Compound 1—{5-[3-(4,6-Difluoro-1H-benzoimidazol-2-yl)-1H-indazol-5-yl]-4-methyl-pyridin-3-ylmethyl}-ethyl-amine

Triethyl silane (976 mg, 8.40 mmol) and trifluoroacetic acid (12.9 mL, 168 mmol) were added to a solution of If (2.02 g, 3.36 mmol) in dichloromethane (12.9 mL). The mixture was stirred at room temperature for 3.5 hours. The volatiles were removed by rotary evaporation, and the residue treated with cyclohexane (10 mL) and aqueous ammonium hydroxide (2 N, 20 mL). After vigorous stirring for 15 minutes, a pink precipitate forms, which was collected by suction filtration. The filtrate was extracted with ethyl acetate (3×50 mL). The combined organic extracts were dried over magnesium sulfate, filtered, and concentrated to an orange solid (˜0.4 g). This solid was added to the pink precipitate obtained above and purified by column chromatography (eluting with a mixture of 1% concentrated ammonium hydroxide to 19% absolute ethanol to 80% dichloromethane). The product thus obtained (1.23 g off-white solid) was further purified by trituration from cyclohexane to yield pure 1 (1.09 g, 74%) as an off-white solid: ¹H NMR (DMSO-d₆) δ 13.81 (very br s, 1H), 8.46 (s, 1H), 8.35 (s, 2H), 7.76 (d, J=8.6 Hz, 1H), 7.44 (dd, J=1.3, 8.6 Hz, 1H), 7.17 (m, 1H), 7.07 (t of d, Jₜ=1.5 Hz, J_{d}=10.6 Hz, 1H) 3.78 (s, 2H), 2.63 (q, J=7.1 Hz, 2H), 2.25 (s, 3H), 1.07 (t, J=7.1 Hz, 3H). HRMS [M+H]⁺ calc. 419.1791; found 419.1811. Anal. (C₂₃H₂₀F₂N₆·1.1 H₂O) C, H, N, F.

### Comparative Example 2

### Preparation of Intermediate (1b) 5-Bromo-4-methyl-pyridin-3-ylmethyl)-ethyl-carbamic acid tert-butyl ester

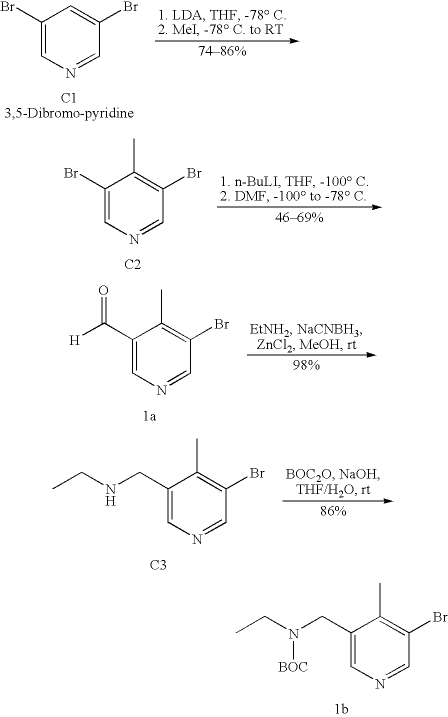

(a) Intermediate 1a: 5-Bromo-4-methyl-pyridine-3-carbaldehyde:

3,5-Dibromo-4-methyl-pyridine (3.8 g, 15.1 mmol) C1 was stirred in dry THF (150 mL) at −100° C. (N₂/ether) under argon. n-Butyllithium (2.5 M in hexanes, 6.2 mL, 15.4 mmol) was added dropwise, and the reaction stirred for 5 minutes DMF (1.8 mL, 23.2 mmol) was added, and the reaction was stirred for 20 minutes at −100° C. and then for 1 hour at −78° C. The reaction was quenched with sat. NH₄Cl and extracted with ether. Organics were washed with brine, dried over Na₂SO₄, and concentrated in vacuo. Purification by silica gel chromatography (20% ethyl acetate/hexanes) gave 2.18 g (72%) of intermediate 1a as a clear oil which slowly solidified. ¹H NMR (300 MHz, CDCl₃) δ 10.25 (s, 1H), 8.84 (s, 1H), 8.83 (s, 1H), 2.76 (s, 3H). Anal. (C₇H₆BrNO) C, H, N.

(b) Intermediate C3—(5-Bromo-4-methyl-pyridin-3-ylmethyl)-ethyl-amine

5-Bromo-4-methyl-pyridine-3-carbaldehyde (6.74 g, 33.7 mmol) was dissolved in methanol (290 mL) under a nitrogen atmosphere. A solution of ethylamine in methanol (2.0 M, 90 ml, 180 mmol) was added dropwise over 30 minutes. Stirring was continued at room temperature for 30 minutes further.

In a separate flask, sodium cyanoborohydride (2.33 g, 37.1 mmol) was dissolved in methanol (150 mL). Anhydrous zinc chloride (2.53 g, 18.5 mmol) was added and stirring continued at room temperature for 20 minutes. This solution (zinc/cyanoborohydride) was then slowly added to the above aldehyde/ethylamine solution. The reaction solution was acidified to pH 4 with 2.0 M HCl in methanol (120 mL), and then stirred at room temperature for 18 hours.

The solvents were removed by rotary evaporation and the residue partitioned between ethyl acetate and 10% aqueous sodium carbonate. The organic extracts were dried over magnesium sulfate and concentrated, affording crude amine C3 (7.36 g, 95%) as an orange oil, which was used in the next step without further purification: ¹H NMR (CDCl₃) δ 8.53 (s, 1H), 8.31 (s, 1H), 3.77 (s, 2H), 2.67 (q, 2H), 2.42 (s, 3H), 1.11 (t, 3H).

(c) Intermediate 1b—(5-Bromo-4-methyl-pyridin-3-ylmethyl)-ethyl-carbamic acid tert-butyl ester

Di-tert-butyl dicarbonate (10.43 g, 47.8 mmol) was added to a solution of crude amine C3 (7.36 g, 32.1 mmol) in THF (400 mL), followed by aqueous sodium hydroxide solution (1.0 M, 101 mL). The biphasic solution was stirred vigorously for 20 hours at room temperature. The solution was partitioned between water and ethyl acetate; the organic extracts were dried over magnesium sulfate, filtered, and concentrated. The crude yellow oil thus obtained was purified by silica gel chromatography (eluting with a gradient of 10% to 30% ethyl acetate in hexanes), yielded bromopyridine 1b (5.37 g, 51%) as a yellow oil: ¹H NMR (CDCl₃) δ 8.58 (s, 1H), 8.22 (s, 1H), 4.47 (s, 2H), 3.17 (br s, 2H), 2.37 (s, 3H), 1.45 (s, 9H), 1.03 (t, 3H).

### Example 3

### Novel Preparation of Intermediate (1b): 5-Bromo-4-methyl-pyridin-3-ylmethyl)-ethyl-carbamic acid tert-butyl ester

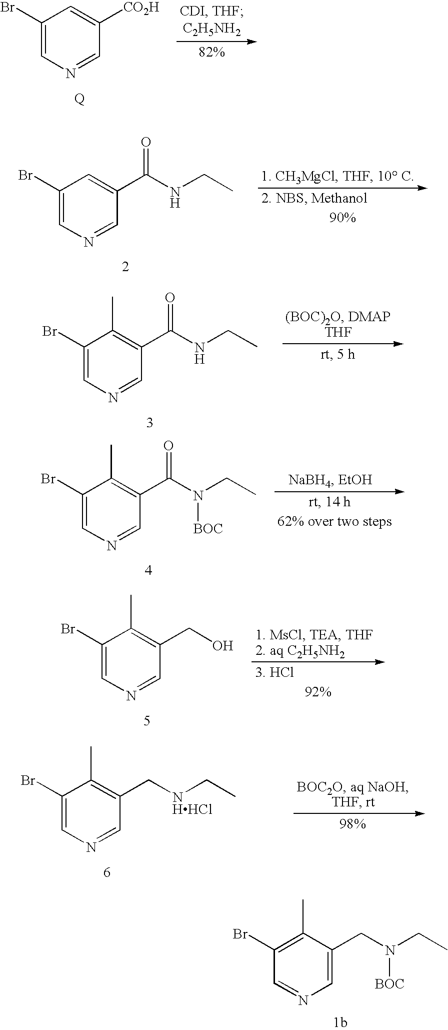 Step 3a: Synthesis of 5-bromo-N-ethyl-nicotinamide 2

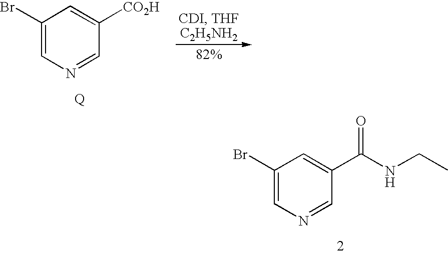

5-bromo-nicotinic acid Q (2 kg, 9.9 M) was dissolved in THF (19 L) and cooled to 0° C. 1,1′-Carbonyldiimidazole (1.76 kg, 10.9 M) was then added over 30 minutes and the reaction mixture was allowed to warm to ambient temperature with additional stirring for 2 hours.

The reaction solution was cooled to −10° C. and ethylamine solution (6.5 L, 13 M) in THF was then added over 20 minutes. The temperature was allowed to rise to 15° C., after which the reaction was stirred at ambient temperature overnight.

The reaction solution was concentrated until solid precipitated out (˜4 L). Distilled water (4 L) was added and the mixture was stirred for 2 h. Solid was collected by filtration, washed with water and dried in vacuo at 45° C. to afford product 2 as white crystals (1.85 kg, 8.08 M, 82%). ¹H NMR (CDCl₃) δ 1.27(t, 3H), 3.52(m, 2H), 8.25(s, 1H), 8.77(s, 1H), 8.86(s, 1H).
Step 3b: Synthesis of 5-bromo-N-ethyl-4-methyl-nicotinamide 3

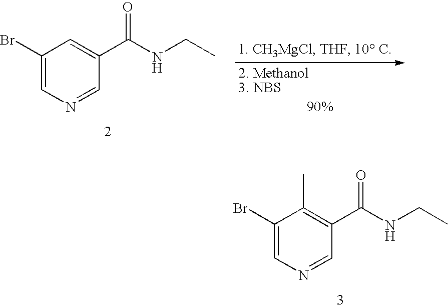

Methylmagnesium chloride (800 ml, 2.40 M) in THF was cooled to 5° C. under nitrogen.

In a separate flask, 5-bromo-N-ethyl-nicotinamide 2 (160.0 g, 699 mmol) was dissolved in anhydrous THF (1.6 L) before addition to the methylmagnesium chloride over 30 minutes via cannula. Temperature of the reaction was maintained between 10° C.-12° C. After addition was complete, the reaction was allowed to warm to 20° C. until a homogeneous yellow-greenish solution was formed, with additional constant stirring for 1.5 hours.

The reaction solution was then cooled to 5° C. and quenched with methanol at a temperature not exceeding 10° C. When addition of methanol was complete, a clear yellow solution was obtained.

N-bromosuccinimide (130.6 g, 733 mmol) was added slowly to the reaction solution at 5° C. (±5° C.), after which the reaction was removed from cooling. The light yellow solution was stirred for additional 30 min. An ammonium chloride solution (314 g, 5.87 M in 1.5 L water) was added to quench the reaction at a temperature not exceeding 20° C., and the reaction mixture was extracted with ethyl acetate. The combined organic solution was concentrated, further extracted with ethyl acetate, and washed with water and aqueous NaOH, and then concentrated to afford 163.3 g of light brown solid 3 (163.3 g, 0.672 mmol, 96%). ¹H NMR (DMSO-d₆) δ 1.12(t, 3H), 2.4(s, 3H), 3.27(m, 3H), 8.49(s, 1H), 8.63(br, 1H), 8.80(s, 1H).
Step 3c: Synthesis of N-BOC-5-bromo-N-ethyl-4-methyl-nicotinamide 4

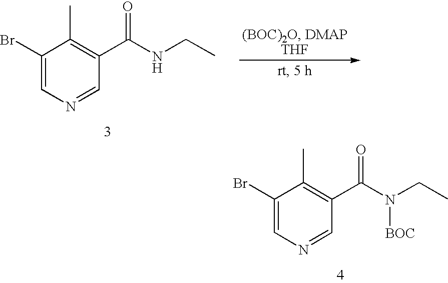

5-bromo-N-ethyl-4-methyl-nicotinamide 3 (156 g, 642 mmol) was dissolved in THF (1.5 L) and Di-t-butyl-dicarbonate (252 g, 1.156 M) was then added to form a clear solution. 4-(Dimethylamino)pyridine (7.83 g, 64.2 mmol) was added and the temperature was allowed to rise to 27° C., with additional continual stirring for 18 hours at room temperature. The reaction mixture was then concentrated, extracted with ethyl acetate and washed with water, before further concentrating to provide a brown oil 4(261 g, 0.763 mmol, 90%). ¹H NMR (CDCl₃) δ 1.20(s, 9H), 1.29(t, 3H), 2.37(s, 3H), 3.92(q, J=7.2 Hz, 2H), 8.25(s, 1H), 8.67(s, 1H).
Step 3d: Synthesis of (5-bromo-4-methyl-pyridin-3-yl)-methanol 5

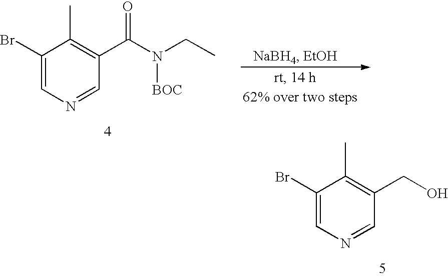

N-BOC-5-bromo-N-ethyl-4-methyl-nicotinamide (3.10 Kg, ˜8.44 M) was dissolved in ethanol (20 L) followed by addition of methanol (1.0 L) and the resulting solution was cooled to 5° C. under nitrogen. Sodium borohydride (480 g, 12.66 M) was added over a period of 30 minutes while maintaining the temperature less than 10° C. The reaction was then stirred overnight at 15±5 ° C.

The reaction was then cooled to 5° C. and a solution of acetic acid (1.3 L) in water (4.0 L) was added slowly at a temperature not exceeding 13° C. The reaction mixture was concentrated at 45° C., extracted with ethyl acetate and washed with aqueous NaOH solution (3.2 L, 2 M). The pH value of the aqueous layer was 7-8.

The mixture was extracted with ethyl acetate and concentrated twice to provide an oil/slurry, which was then suspended in ethyl acetate (1.2 L). The suspension was stirred at ambient temperature for 60 minutes and excess of heptane (12.0 L) was then added slowly. After stirred for 2 h at ambient temperature, the slurry was filtered and rinsed with heptane. The cake was dried in vacuo at 35° C. overnight to afford an off-white solid 5 (1.07 g, 0.00529 mmol, 62%). ¹H NMR (CDCl₃) δ 2.45(s, 3H), 4.74(s, 2H), 8.38(s, 1H), 8.58(s, 1H).
Step 3e: Synthesis of (5-Bromo4-methyl-pyridin-3-ylmethyl)-ethyl-amine 6

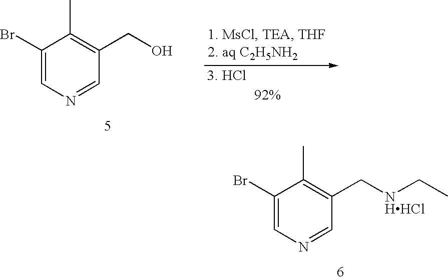

(5-Bromo-4-methyl-pyridin-3-yl)-methanol 5 (95.7 g, 473.8 mmol) was dissolved in THF (1.3 L) and the resulting slurry was cooled to −15° C.

In a separate flask a solution of triethylamine (71.8 g, 710.7 mmol) in THF (61 ml) was prepared.

In yet another flask a solution of methanesulfonyl chloride (65.1 g, 568.6 mmol ) in THF (116 ml) was prepared.

Both solutions were added in separate funnels to the reaction at the same rate at a temperature between −15° C. to −5° C. The reaction mixture was then stirred for an additional 15 minutes at −15° C.

70% ethylamine solution (305 g) in water was added slowly via additional funnel at a temperature between −15° C. to 0° C. The solution was stirred at 0° C. for 2 hours and then was allowed to warm up to the ambient temperature over 16 hours.

The solvent was removed to give a brown oil (312 g). The residue was then extracted with ethyl acetate and washed with water, dried and again extracted with ethyl acetate (600 ml) before being dried again. The residual oil/slurry was again dissolved in ethyl acetate (500 ml) and the solution was cooled to 12° C. HCl in dioxane (4M, 124 ml, 1.05 equiv.) was added slowly and pink solid precipitated out. The mixture was stirred for 1 hour at the ambient temperature, then filtered and washed with EtOAc. The cake was air-dried overnight to afford a pink solid (116 g, 439 mmol, 92%). ¹H NMR (DMSO-d₆) δ 1.28(t, 3H), 2.51 (s, 3H), 3.06(m, 2H), 4.25(m, 2H), 8.65(s, 1H), 8.72(s, 1H), 9.38(s, br, 2H).
Step 3f: Synthesis of (5-Bromo-4-methyl-pyridin-3-ylmethyl)-ethyl-carbamic acid t-butyl ester (Intermediate 1b)

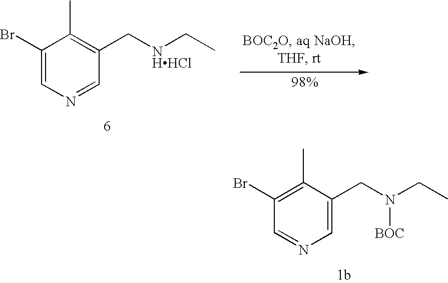

The hydrochloride salt of (5-Bromo-4-methyl-pyridin-3-ylmethyl)-ethyl-amine 6 (43.53 g, 165 mmol) was suspended in THF (500 ml) and dissolved in aqueous NaOH (165 ml, 3 M).

Di-t-butyl-dicarbonate was added in several portions at a temperature of 25° C. (±5° C.) and the reaction was then stirred at room temperature for 2-4 hours.

DMAP (1.0 g, 8 mmol) was added and the reaction mixture was stirred for 4 additional hours with solid precipitating out. MTBE (200 ml) was added followed by addition of water (200 ml) to dissolve the precipitate.

The aqueous phase was separated and the organic phase was dried. The reaction mixture was extracted with EtOAc (400 ml) and 5% aqueous ammonium chloride solution (400 ml), and washed sequentially with water, 2% aqueous sodium bicarbonate solution, and again water. The solution was then concentrated to dryness, extracted with ethyl acetate, and again concentrated. The residue was further dried in vacuo to afford the product as an oil 1b (52.86 g, 161 mmol, 98%) ¹H NMR (300 MHz, CDCl₃) δ 1.03(t, 3H), 1.45(s, 9H), 2.37(s, 3H), 3.17(s, br, 2H), 4.47(s, 2H), 8.22(s, 1H), 8.58(s, 1H).

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

We claim:

1. A method of preparing a compound of formula 1b 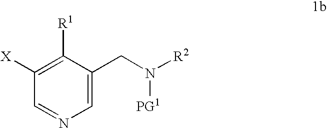
wherein X is a halo;
R¹ and R² are, independently, alkyl, alkenyl, alkynyl, cycloalkyl or aryl;
PG¹ is a protecting group selected from tert-butoxycarbonyl (“BOC”), benzyloxycarbonyl (“CBZ”), CH₂C₆H₅ (“Bn”), 2-(trimethylsilyl)ethoxymethyl (“SEM”), tetrahydropyran (“THP”), trimethylsilyl (“TMS”);
comprising the step of reacting a compound of formula 6 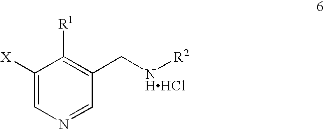
with PG¹ and a base in a suitable solvent;
and further comprising the step of preparing a compound of formula 6, by reacting a compound of formula 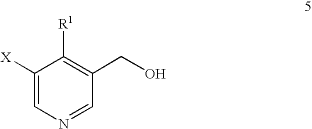
with R²NH₂, an acid chloride and a base in a suitable solvent.

2. The method of claim 1, further comprising the step of preparing a compound of formula 5 by reacting a compound of formula 4 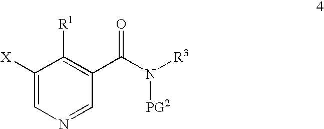
wherein PG² is selected from BOC, CBZ, Bn, SEM, THP or TMS;
R³ is selected from an alkyl, alkenyl, alkynyl, cycloalkyl or aryl;
with a reducing agent and an alcohol.

3. The method of claim 2, further comprising the step of preparing a compound of formula 4 by reacting a compound of formula 3 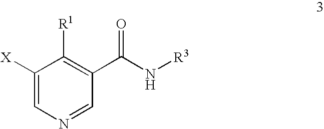
with a PG² and a base in a suitable solvent.

4. The method of claim 3, further comprising the step of preparing a compound of formula 3 by reacting a compound of formula 2 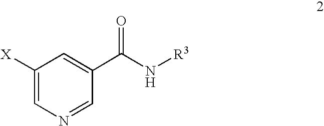
with an alkylating agent and an oxidizing reagent in a suitable solvent.

5. The method of claim 4, further comprising the step of preparing a compound of formula 2 by reacting a compound of formula Q 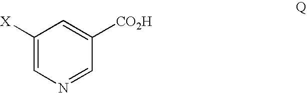
with R²NH₂ and 1,1′-carbonyldiimidazole in a suitable solvent.

6. The method of claim 5, wherein R¹ is methyl, R² is ethyl and the alkylating agent is a methylating agent.

7. The method of claim 6, wherein R³ is ethyl.

8. The method of claim 1, wherein the acid chloride is methanesulfonyl chloride and the aqueous base is triethylamine.

9. The method of claim 7, wherein X is Br.

10. The method of claim 3, wherein PG¹ and PG² are BOC.

11. The method of claim 5, wherein the suitable solvent is selected from THF, CH₂Cl₂, MTBE, toluene or a combination thereof.

12. The method of claim 10, wherein the suitable solvent is THF.

13. The method of claim 3, wherein the alcohol is selected from methanol, ethanol or a sequential combination thereof.

14. The method of claim 2, wherein the reducing agent is selected from selected from NaBH₄, LiBH₄ or LiAlH₄.

15. The method of claim 13, wherein the reducing agent is NaBH₄.

16. The method of claim 3, wherein the base is selected from aqueous NaOH, 4-dimethylaminopyridine, N,N-diisopropylethylamine or triethylamine.

17. The method of claim 15, wherein the base is 4-dimethylaminopyridine.

18. The method of claim 6, wherein the methylating agent is CH₃MgCl, CH₃Li, (CH₃)₂CuLi or CH₃ZnCl.

19. The method of claim 17, wherein the methylating agent is CH₃MgCl.

20. The method of claim 4, further comprising a step of adding a quenching solvent to compound 2 and the alkylating agent; wherein the quenching solvent is aqueous ammonium chloride or an alcohol selected from methanol, ethanol, or a sequential combination thereof.

21. The method of claim 4, wherein the oxidizing reagent is selected from N-bromosuccinimide or N-chlorosuccinimide.

22. The method of claim 21, wherein the oxidizing reagent is N-bromosuccinimide.
